(19) United States
(12) Patent Application Publication
(10) Pub. No.: US 2017/0258350 A1
(43) Pub. Date: Sep. 14, 2017

(54) Heart Murmur Detection Device and Method Thereof

(71) Applicant: Hong Wen FANG, Taipei City (TW)

(72) Inventor: Hong Wen FANG, Taipei City (TW)

(21) Appl. No.: 15/066,909

(22) Filed: Mar. 10, 2016

(51) Int. Cl. A61B 5/0452; A61B 5/0404; A61B 5/0205; A61B 5/00; A61B 7/02

(52) U.S. Cl. CPC A61B 5/0452; A61B 5/7203; A61B 7/026; A61B 5/0205; A61B 5/742; A61B 5/0404

(57) ABSTRACT

The present invention provides a heart murmur detection device. The heart murmur detection device includes: an ECG signal detection unit for detecting an ECG signal from a heart of a user; a heart beat detection unit for detecting the frequency of the ECG signal; a plurality of sound receiving units for receiving a plurality of sound signals from the heart of the user; a signal transforming unit for transforming the sound signals into a plurality of electric phonic signals, and for retrieving the electric phonic signals on the basis of the ECG signal; and a signal processing unit for determining a heart murmur generating position. Moreover, the present invention further provides a heart murmur detection method.

### BACKGROUND OF THE INVENTION

1. Field of the Invention

The present invention relates to a detection device and method thereof, and relates in particular to a heart murmur detection device and method thereof.

2. The Prior Arts

Heart murmurs are sounds produced by blood flow within the heart of a patient. These heart murmurs may be caused by abnormal blood flow in the heart, or may be caused by abnormalities of the heart or blood vessels. Heart murmurs can generally be classified into two main groups: the first group is functional murmurs, and the second group is pathologic murmurs. The reasons for the occurrence of functional murmurs may include peripheral pulmonary stenosis, patent ductus arteriosus, tricuspid regurgitation and the like. However, the reasons for the occurrence of pathological murmurs include ventricular septal defect, valvular stenosis or regurgitation, atrial or ventricular compartments chipping and the like. Functional murmurs are usually benign and harmless. However, pathological murmurs are usually caused by abnormalities of the heart and blood vessels, and usually offer indications of heart diseases.

The intensity of the heart murmurs can be classified into 6 grades, with grade 1 being the weakest, and grade 6 being the strongest. A grade 1 heart murmur refers to sounds besides a patient's heartbeat which are heard by a doctor using a stethoscope in a quiet environment. A grade 4 heart murmur refers to a noise having a vibration that can be felt through touching. Moreover, a grade 6 heart murmur refers to noise that can be heard without attaching the stethoscope closely to the chest of a patient. The examination of heart murmurs is mainly through a stethoscope. When the stethoscope is firmly attached to the chest of the patient by the doctor, the doctor is able to make a preliminary diagnosis of any heart related diseases, on the basis of the duration, intensity and timing of the sounds. Such diseases may include arrhythmia, inflammation of the pericardium, valvular stenosis, prolapse or regurgitation, ventricular septal defect, arteriovenous fistula and the like.

The traditional stethoscopes are mainly made up of materials such as copper, rubber tubes, spring plate, head set and the like. Stethoscopes can be classified as single-use stethoscope, dual-use stethoscope, tri-use stethoscope, vertical stethoscope and multi-function stethoscope, which are all used with different patients in different situations. However, following the advancement of medical technologies, the function of stethoscopes today is becoming more and more sophisticated; and electronic stethoscopes are emerging. The traditional stethoscopes are mainly used for sound collection, and they do not have the effect of sound amplification. However, electronic stethoscopes have excellent function of sound volume digital amplification, and thus enabling the heart sounds of the patient to be clearly heard.

However, the examination of heart murmur in a patient is normally interfered by sounds from the other organs, for example the breathing sounds of the lungs. A sound is given out when the general person breathes, however, a noise can be heard when a person with breathing difficulty breathes. Furthermore, when pulmonary obstructive hydrocephalus occurs in the patient, different noises can be heard. The occurrence of a noise often interferes with the interpretation of noises by a doctor. As such, how to solve the problem of not easily distinguishing a heart murmur in a patient is in fact a technical question that is desired to be solved.

### SUMMARY OF THE INVENTION

In view of the drawbacks of the prior art, one of the objectives of the present invention is to provide a heart murmur detection device, in order to solve the problem of not being able to easily interpret/distinguish a heart murmur in a patient during examination, due to the interference by other noises from other organs.

In order to achieve the above-mentioned objectives, the present invention includes a heart murmur detection device, which includes: an ECG signal detection unit, which may include at least two electrodes, for detecting an ECG signal from the heart of a user; a heart beat detection unit, which may electrically connect to the ECG signal detection unit, and may retrieve a frequency of the ECG signal; a plurality of sound receiving units, which may receive a plurality of sound signals from the heart of the user; a signal transforming unit, which may electrically connect to the plurality of sound receiving units and the heart beat detection unit, whereby the signal transforming unit may transform the plurality of sound signals into a plurality of electric phonic signals, and may retrieve the plurality of electric phonic signals on the basis of the frequency of the ECG signal; and a signal processing unit, which may be electrically connected to the signal transforming unit, and may determine a heart murmur generating position on the basis of the plurality of electronic phonic signals.

Preferably, the signal processing device may determine the heart murmur generating position on the basis of the intensity of the plurality of electric phonic signals.

Preferably, the signal processing unit may determine the heart murmur generating position of the patient on the basis of the direction formed by the largest electric phonic signal and the smallest electric phonic signal of the plurality of electric phonic signals.

Preferably, the signal processing unit may perform a spectrum analysis based on the largest electric phonic signal, so as to obtain a heartbeat waveform and a heart murmur waveform.

Preferably, the present invention may further include a display unit, which may be electrically connected to the signal processing unit, and may display the heartbeat waveform and the heart murmur waveform.

Preferably, the plurality of sound receiving units may amplify the plurality of sound signals which are generated from the heart of the user.

Preferably, the present invention may further include a plurality of indicator lights, which may correspondingly display the intensity of the plurality of electric phonic signals and the heart murmur generating position.

Preferably, the heart beat detection unit, the signal transforming unit and the signal processing unit may be integrated into an integrated circuit.

Preferably, the heart murmur detection device may be a handheld module.

In addition, the present invention also provides a method for detecting heart murmur, so as to solve the problem of not being able to easily distinguish a heart murmur in a patient during examination, due to the interference by other noises from other organs.

In order to achieve the above-mentioned objective, the heart murmur detection method of the present invention includes the following steps: Step A: an ECG signal from the heart of a user may be detected by means of an ECG detection unit; Step B: the frequency of the ECG signal may be retrieved by means of a heart beat detection unit; Step C: a plurality of sound signals may be received from the heart of the user by means of a plurality of sound receiving units; Step D: the plurality of sound signals may be transformed into a plurality of electric phonic signals by means of a signal transforming unit, and the plurality of electric phonic signals may be retrieved based on the frequency of the ECG signal; and Step E: a heart murmur generating position may be determined by means of a signal processing unit based on plurality of electric phonic signals.

The present invention firstly integrates an ECG signal detection unit and a sound receiving unit having the function of a stethoscope, analyzing the signals that are received by the ECG signal detection unit and the sound receiving unit, and then determining the heart murmur generating position by means of electric phonic signals. As such, the heart murmur detection device and method thereof of the present invention can effectively determine the heart murmur generating position in a patient.

### BRIEF DESCRIPTION OF THE DRAWINGS

The present invention can be understood in more detail by reading the subsequent detailed description in conjunction with the examples and references made to the accompanying drawings, wherein:

FIG. 1 is a system block diagram in accordance with a preferred embodiment of the present invention.

FIG. 2 is a schematic diagram in accordance with a preferred embodiment of the present invention.

FIG. 3 shows a schematic diagram in accordance with another preferred embodiment of the present invention.

FIG. 4 is a flow chart showing the method in accordance with a preferred embodiment of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

It should be noted that the structures, proportions, sizes and the like of the drawings of the current specification are only for depicting the disclosures of the present invention, to enable easy reading and understanding by persons skilled in the art, and these are not meant to be conditions that limit the present invention. As such, the drawings are not technologically meaningful. Any modifications of the structure, proportion relationships or any adjustments of the sizes should fall within the scope that is covered by the technical content of the present invention, without affecting the effects produced by the present invention and without affecting the goals of the present invention.

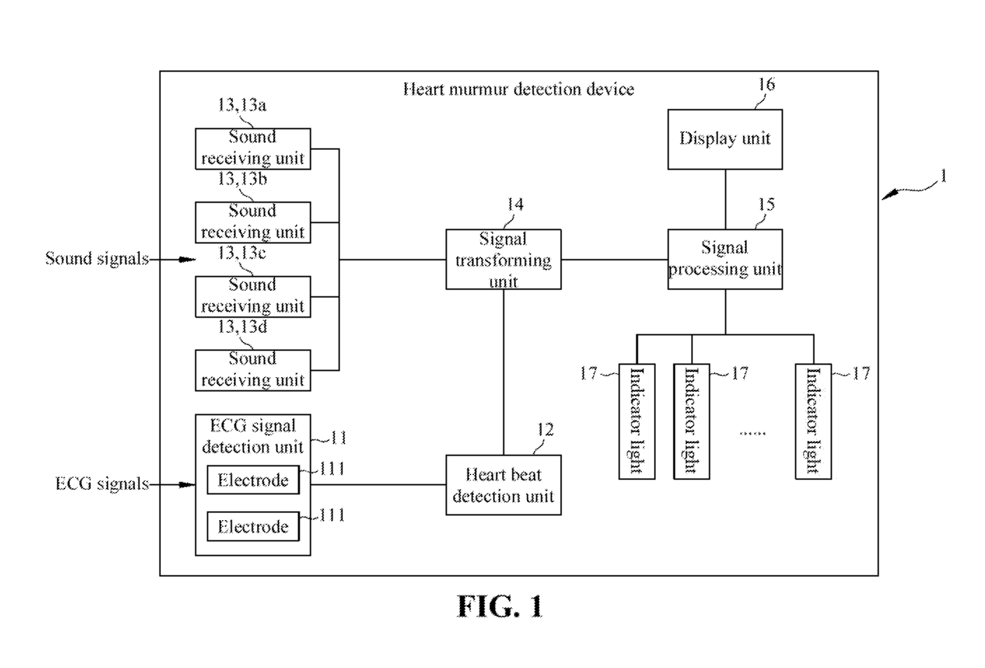

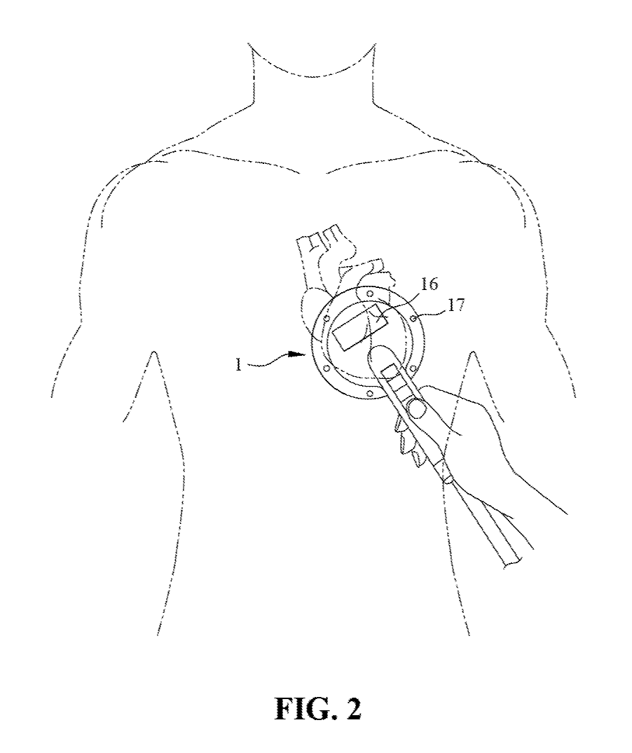

Please refer to FIG. 1 and FIG. 2 together. FIG. 1 is a system block diagram in accordance with an embodiment of the present invention. FIG. 2 is a schematic diagram in accordance with an embodiment of the present invention. As shown in FIG. 1 and FIG. 2, the heart murmur detection device 1 of the present invention includes the following: an ECG signal detection unit 11, which may include at least two electrodes 111; the ECG signal detection unit 11 may detect an ECG signal from the heart of a user; a heart beat detection unit 12, which may be electrically connected to the ECG signal detection unit 11, so as to retrieve the frequency of the ECG signal; a plurality of sound receiving units 13, which may receive the plurality of sound signals from the heart of a user; a signal transforming unit 14, which may be electrically connected to the plurality of sound receiving units 13 and the heart beat receiving unit 12, whereby, the signal transforming unit 14 may transform the plurality of sound signals into the plurality of electric phonic signals, and the plurality of electric phonic signals may be retrieved on the basis of the frequency of the ECG signal. Also, the heart murmur detection device of the present invention may also include a signal processing unit 15, which may be electrically connected to the signal transforming unit 14. A heart murmur generating position in the patient may be determined by the signal processing unit 15 on the basis of the plurality of electric phonic signals.

In accordance with a preferred embodiment of the present invention, the ECG signal detection unit 11 may include multiple electrodes 111, for example three or four electrodes (not shown in the drawings), so as to increase the accuracy for detecting the ECG signals.

In accordance with a preferred embodiment of the present invention, the heart murmur generating position may be determined by the signal processing unit 15 on the basis of the intensity of the plurality of electric phonic signals. For example, the sound receiving unit 13a may receive the strongest sound signal, after the plurality of sound signals are transformed into the electric phonic signals. The signal processing unit 15 may determine the actual location of the sound receiving unit 13a, and may receive the strongest sound signal. As such, the heart murmur generating position may be found close to the sound receiving unit 13a.

In accordance with a preferred embodiment of the present invention, the signal processing unit 15 may determine the heart murmur generating position based on the direction of the largest electric phonic signal and the smallest electric phonic signals from the plurality of electric phonic signals. For example, the sound receiving unit 13a may receive the largest sound signal, and the sound receiving unit 13b may receive the smallest sound signal. After the largest or smallest sound signals are transformed into the plurality of electric phonic signals, the signal processing unit 15 may determine that the strongest sound signals may be received between the sound receiving unit 13a and the sound receiving unit 13b. Therefore, the heart murmur generating position may be found in between the sound receiving unit 13a and the sound receiving unit 13b.

In accordance with a preferred embodiment of the present invention, the signal processing unit 15 may perform an analysis of the frequency spectrum based on the largest electric phonic signals, so as to isolate a heartbeat waveform and a heart murmur waveform.

In accordance with a preferred embodiment of the present invention, the heart murmur detection device of the present invention may further include a display unit 16. The display unit 16 may be electrically connected with the signal processing 15. The display unit 16 may display the heartbeat waveform and the heart murmur waveform. The two waveforms may be displayed in different colors by the display unit 16, such that the waveforms may be easily determined by the reader.

In accordance with a preferred embodiment of the present invention, the plurality of sound receiving units 13 may amplify the plurality of sound signals from the heart of a user, so as to increase the accuracy of distinguishing the heart murmur.

In accordance with a preferred embodiment of the present invention, the heart murmur detection device of the present invention may further include a plurality of indicator lights, which may correspondingly display the intensity of the plurality of electric phonic signals and the heart murmur generating position of the user. The plurality of indicator lights 17 may correspond to the positions of the sound receiving units 13. When one of the sound receiving units receives the strongest sound signals, the indicator light 17 that corresponds to that particular sound receiving unit 13 may correspondingly light up, so as to indicate the heart murmur generating position of the user.

In accordance with a preferred embodiment of the present invention, the heartbeat detection unit 12, the signal transforming unit 14 and the signal processing unit 15 may be integrated as an integrated circuit.

In accordance with a preferred embodiment of the present invention, the heart murmur detection device 1 may be a handheld module, but is not limited to this. Whereby, the ECG signal detection unit 11 and the plurality of sound receiving units 13 may be integrated into one end of the handheld module. The display unit 16 and the plurality of indicator lights 17 may be installed on a surface of the handheld module. While the handheld module is being used, it may be placed near the position of the heart of the user, whereby preferably, the handheld module may be placed directly above the heart of the user.

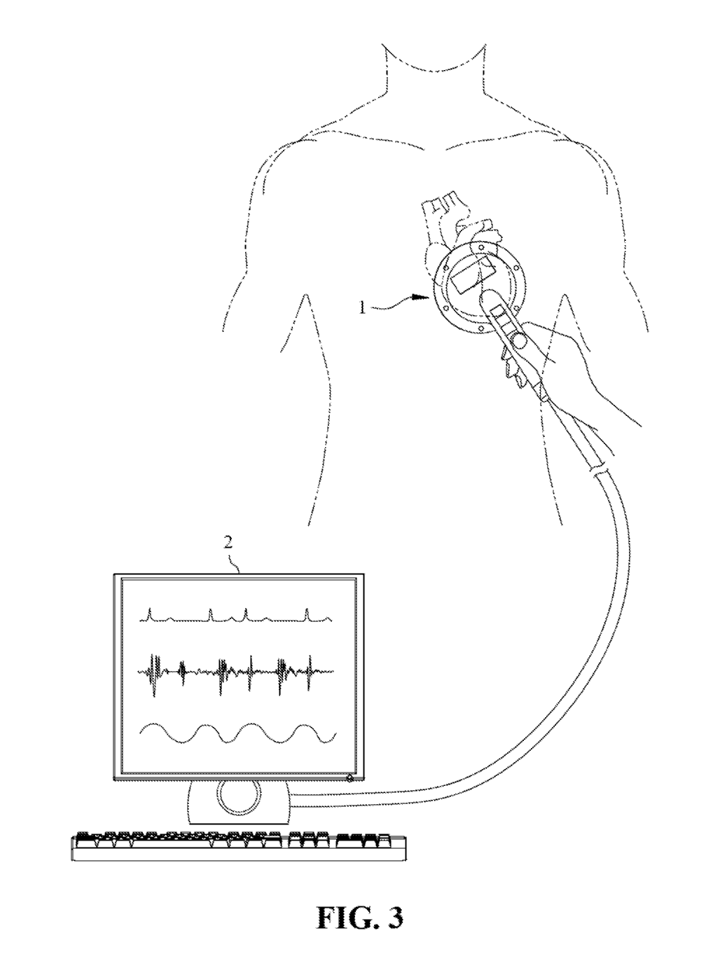

In accordance with a preferred embodiment of the present invention, the heart murmur detection device 1 may be connected externally to a display screen 2, such that the ECG signals, electric phonic signals, heartbeat waveform and heart murmur waveform may be clearly displayed, as shown in FIG. 3.

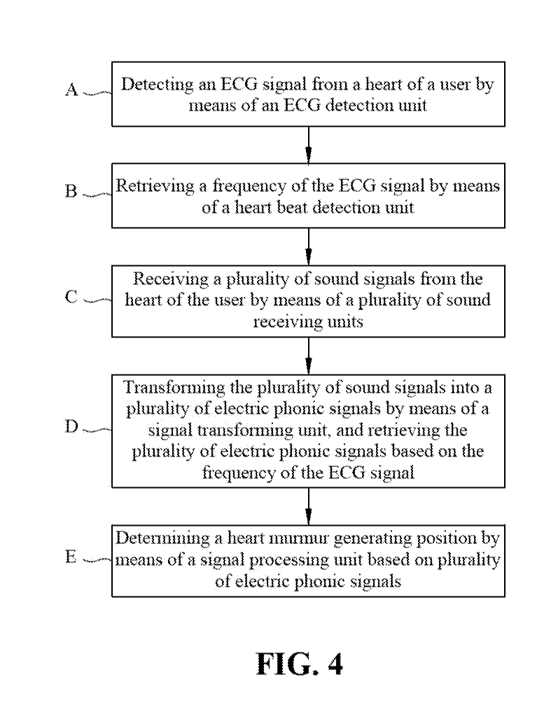

In addition, the present invention also provides a method for detecting heart murmur. Please refer to FIG. 1 and FIG. 4 together. FIG. 4 is a flow chart showing the method of the present invention. As shown in the FIGs, the method for detecting a heart murmur of the present invention may include the following steps: Step A: an ECG signal from the heart of a user may be detected by means of an ECG detection unit 11; Step B: the frequency of the ECG signal may be retrieved by a heartbeat detection unit 12; Step C: the plurality of sound signals from the heart of the user may be received by the plurality of sound receiving units; Step D: the plurality of sounds signals may be transformed into the plurality of electric phonic signals by the signal transforming unit 14, and the plurality of electric phonic signals may be retrieved based on the frequency of the ECG signal; and Step E: a heart murmur generating position may be determined by means of a signal processing unit 15 based on the plurality of electric phonic signals.

The present invention establishes the heart murmur generating position of a user by firstly integrating an ECG signal detection unit 11 and a sound receiving unit 13 that has the function of a stethoscope, analyzing the signals that are received by the ECG signal detection unit 11 and the sound receiving unit 13, and then determining the heart murmur generating location by means of the plurality of electric phonic signals. As such, the heart murmur detection device and method thereof of the present invention is able to effectively determine the heart murmur generating position in a patient.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A heart murmur detection device, comprising:
an ECG signal detection unit, comprising at least two electrodes, and detecting an ECG signal from a heart of a user;
a heart beat detection unit, electrically connecting to the ECG signal detection unit, and retrieving a frequency of the ECG signal;
a plurality of sound receiving units, receiving a plurality of sound signals from the heart of the user;
a signal transforming unit, electrically connecting to the plurality of sound receiving units and the heart beat detection unit, wherein the signal transforming unit transforms the plurality of sound signals into a plurality of electric phonic signals, and retrieving the plurality of electric phonic signals on the basis of the frequency of the ECG signal; and
a signal processing unit, electrically connecting to the signal transforming unit, and determining a heart murmur generating position on the basis of the plurality of electronic phonic signals.

2. The heart murmur detection device of claim 1, wherein the signal processing device determines the heart murmur generating position on the basis of the intensity of the plurality of electric phonic signals.

3. The heart murmur detection device of claim 1, wherein the signal processing unit determines the heart murmur generating position on the basis of the direction formed by a largest electric phonic signal and a smallest electric phonic signal of the plurality of electric phonic signals.

4. The heart murmur detection device of claim 1, wherein the signal processing unit performs a spectrum analysis based on the largest electric phonic signal, so as to obtain a heartbeat waveform and a heart murmur waveform.

5. The heart murmur detection device of claim 1, further comprising a display unit, electrically connecting to the signal processing unit, and displaying the heartbeat waveform and the heart murmur waveform.

6. The heart murmur detection device of claim 1, wherein the plurality of sound receiving units amplify the plurality of sound signals which are generated from the heart of the user.

7. The heart murmur detection device of claim 1, further comprising a plurality of indicator lights, correspondingly displaying the intensity of the plurality of electric phonic signals and the heart murmur generating position.

8. The heart murmur detection device of claim 1, wherein the heart beat detection unit, the signal transforming unit and the signal processing unit are integrated into an integrated circuit.

9. The heart murmur detection device of claim 1, wherein the heart murmur detection device is a handheld module.

10. A method for detecting heart murmur, comprising the steps of:
detecting an ECG signal from a heart of a user by means of an ECG detection unit;
retrieving a frequency of the ECG signal by means of a heart beat detection unit;
receiving a plurality of sound signals from the heart of the user by means of a plurality of sound receiving units;
transforming the plurality of sound signals into a plurality of electric phonic signals by means of a signal transforming unit, and retrieving the plurality of electric phonic signals based on the frequency of the ECG signal; and
determining a heart murmur generating position by means of a signal processing unit based on plurality of electric phonic signals.
